(19) United States
(12) Patent Application Publication
(10) Pub. No.: US 2009/0180956 A1
(43) Pub. Date: Jul. 16, 2009

(54) DIAGNOSTIC FOR EVALUATING GASTRIC EVACUATION FUNCTION AND/OR SMALL INTESTINAL ABSORPTION FUNCTION

(76) Inventors: Asuka Ito, Tokyo (JP); Tadashi Kohno, Tokyo (JP); Isaburo Hosoi, Tokyo (JP); Junko Hirayama, Tokyo (JP); Katsuhiro Aoyama, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/298,516

(22) PCT Filed: Apr. 25, 2007
(86) PCT No.: PCT/JP2007/058908

§ 371 Date: Oct. 24, 2008

(30) Foreign Application Priority Data
May 1, 2006 (JP) 2006-127240

(51) Int. Cl. A61K 51/04; A61P 1/00

(52) U.S. Cl. 424/1.81

(57) ABSTRACT

The present invention provides a diagnostic agent for evaluating gastric emptying function and/or small intestinal absorptive capacity which imposes less burden to subjects and which is capable of obtaining objective test results in a short period of time. A diagnostic agent for evaluating gastric emptying function and/or small intestinal absorptive capacity, comprising succinic acid labeled with a stable isotope ¹³C or a pharmaceutically acceptable salt thereof.

### TECHNICAL FIELD

The present invention relates to a diagnostic agent for evaluating gastric emptying function and/or small intestinal absorptive capacity.

### BACKGROUND ART

As methods for testing absorption in the small intestine, xylose absorption test, protein absorption test, lipid absorption test, vitamin absorption test (Non-Patent Document 1) and so one have been known. Xylose absorption test is a method in which urinary excretion of xylose is quantified after xylose intake. This method has a drawback that it is easily affected by renal function, and yet the operations of this method are complicated because 5-hour urine collection is necessary. Protein absorption test is a method in which protein absorption ratio is calculated from protein intake and excretion of protein in stool. This method has a drawback that it is affected by indigestion, and yet the operations of this method are complicated because stool collection is necessary. Lipid absorption test is a method in which the amount of lipid in stool is quantified. This method has a drawback that it is affected by indigestion, and yet the operations of this method are complicated because stool collection is necessary. Vitamin absorption test is a method in which urinary radioactivity is quantified after intake of a radioisotope-labeled vitamin and then the urinary excretion ratio of the vitamin is calculated. This method has a drawback that it is easily affected by renal function and a danger that radio-exposure is unavoidable, and yet the operations of this method are complicated because 24-hour urine collection is necessary.

If there is a simple and less invasive method for evaluating small intestinal absorptive capacity, such a method may be applicable to the judgment of the time of shifting from intravenous feeding to enteral feeding, which judgment is often made empirically. Thus, it will be possible to prevent the introduction of enteral feeding at a too early stage when gastrointestinal movement has not been recovered sufficiently (which inevitably forces to return to intravenous feeding), and to assure smooth introduction of enteral feeding. It is also believed that such a method is useful as an evaluation indicator for gastrointestinal surgery.

As methods for testing gastric emptying, methods such as a marker method, an acetaminophen method, an isotope method, a method by helical CT or a method by MRI (Non-Patent Document No. 2) are known. The marker method is a method in which a radiopaque marker is administered, and then x-ray images are taken with the passage of time and evaluated. This method has a danger that radio-exposure is unavoidable. The acetaminophen method is a method in which, after intake of acetaminophen together with test meal, blood samples are taken with the passage of time and acetaminophen concentrations therein are quantified. This method requires complicated operations for collecting blood samples repeatedly, and yet this method is inconvenient in a point that the test result can not be obtained in situ because the quantification of acetaminophen is performed by HPLC analysis. The isotope method is a method in which, after intake of a radioisotope-labeled test meal, the radioactivity in the stomach is measured with the passage of time with a gamma camera. This method has a danger that radio-exposure is unavoidable. The method by helical CT requires a large-scaled and expensive apparatus and also has a danger that radio-exposure is unavoidable. The method by MRI requires a large-scaled and expensive apparatus, and yet this method is inconvenient because the subject must be restrained at supine position for 60 minutes.

If there is a simple method for evaluating gastric emptying, it will become possible to evaluate routinely, for example, the gastric emptying of diabetic patients.

In diabetic patients who need insulin treatment, insulin is administered at a specific time prior to meal. When gastric emptying is delayed, delivery of food from the stomach to the small intestine is delayed. As a result, insulin exerts its effect before food digestion/absorption and rise in blood sugar take place, which causes hypoglycemia. Difficulty in glycemic control in diabetic patients with this delayed gastric emptying has become an issue. Therefore, knowing the timing of gastric emptying in diabetic patients leads to knowing the appropriate timing of insulin administration and is very significant for achieving glycemic control.

Under such circumstances, development of a method for evaluating gastric emptying function or small intestinal absorptive capacity is desired which imposes less burden to subjects and is capable of obtaining objective test results in a short period of time.
[Non-Patent Document 1] Modern Clinical Function Test (II), Japanese Journal of Clinical Medicine Vol. 55 (1997) extra issue, Manual of Clinical Laboratory Medicine Revised 32nd Ed, (published by Kanehara Co., & Ltd.), p. 1309
[Non-Patent Document 2] Modern Clinical Function Test (II), Japanese Journal of Clinical Medicine Vol. 55 (1997) extra issue, Manual of Clinical Laboratory Medicine Revised 32nd Ed. (published by Kanehara Co., & Ltd.), p. 1308

### DISCLOSURE OF THE INVENTION

### Problem for Solution by the Invention

It is an object of the present invention to provide a diagnostic agent for evaluating gastric emptying function and/or small intestinal absorptive capacity which imposes less burden to subjects and is capable of obtaining objective test results in a short period of time.

### Means to Solve the Problem

The present inventors have found that it is possible to detect abnormalities in gastric emptying function and small intestinal absorptive capacity by administering to a subject succinic acid labeled with ¹³C and then measuring ¹³CO₂ discharged into the breath. Thus, the present invention has been achieved.

The subject matters of the present invention are as follows.
(1) A diagnostic agent for evaluating gastric emptying function and/or small intestinal absorptive capacity, comprising succinic acid labeled with a stable isotope ¹³C or a pharmaceutically acceptable salt thereof.
(2) The diagnostic agent of (1) above, wherein the succinic acid is selected from the group consisting of [1,4-¹³C] succinic acid, [1-¹³C] succinic acid, [2,3-¹³C] succinic acid, [2-¹³C] succinic acid and [U-¹³C] succinic acid.
(3) The diagnostic agent of (1) or (2) above, which is a preparation for oral administration.
(4) The diagnostic agent of any one of (1) to (3) above, which is for use in a breath test where ¹³CO₂ discharged into the breath after administration is measured.
(5) The diagnostic agent of any one of (1) to (4) above, which detects diseases associated with abnormalities in gastric emptying function and/or small intestinal absorptive capacity.
(6) The diagnostic agent of (5) above, which detects functional dyspepsia.
(7) The diagnostic agent of (5) above, which detects small intestinal epithelium disorder caused by fasting, drug-induced enteritis, short bowel syndrome or irritable bowel syndrome.
(8) The diagnostic agent of any one of (1) to (4) above, which evaluates the gastric emptying function of a patient who underwent a gastrointestinal surgery, a patient with diabetes, a patient with functional dyspepsia or a patient with reflux esophagitis.
(9) The diagnostic agent according to any one of (1) to (4) above, which is for use in judging anastomotic leak in a patient who underwent a gastrointestinal surgery.
(10) The diagnostic agent of any one of (1) to (4) above, which is for use in judging the appropriateness of shifting from parenteral nutrition to enteral nutrition or oral feeding.
(11) Use of succinic acid labeled with a stable isotope ¹³C or a pharmaceutically acceptable salt thereof for manufacturing a diagnostic agent for evaluating gastric emptying function and/or small intestinal absorptive capacity.
(12) A method of evaluating gastric emptying function and/or small intestinal absorptive capacity, comprising administering to a subject succinic acid labeled with a stable isotope ¹³C or a pharmaceutically acceptable salt thereof.

### Effect of the Invention

According to the present invention, diagnosis of gastric emptying function and/or small intestinal absorptive capacity has become possible which imposes less burden to subjects and is capable of obtaining objective test results in a short period of time. The diagnostic agent of the present invention is cheap and has no adverse effect. Thus, the diagnostic agent of the present invention can be used safely.

The present specification encompasses the contents described in the specification and/or the drawings of Japanese Patent Application No. 2006-127240 based on which the present patent application claims priority.

### BRIEF DESCRIPTION OF THE DRAWINGS

FIG. 1 shows the time course of the increase ratio of ¹³C concentration (Δ¹³C (‰)) in exhaled CO₂ after administration of [1,4-¹³C]-succinic acid into the duodenum. [1,4-¹³C]-succinic acid was administered into the duodenum of pylorus-ligated rats and non-treated rats at 0 minute.

FIG. 2 shows the time course of the increase ratio of ¹³C concentration (Δ¹³C (‰)) in exhaled CO₂ after administration of [1,4-¹³C]-succinic acid into the duodenum. [1,4-¹³C]-succinic acid was administered into the duodenum of MTX-administered rats and control rats at 0 minute. Bars indicate SD.

FIG. 3 shows the time course of the increase ratio of ¹³C concentration (Δ¹³C (‰)) in exhaled CO₂ after administration of [1,4-¹³C]-succinic acid into the femoral vein. [1,4-¹³C]-succinic acid was administered into the femoral vein of nSTZ rats and control rats at 0 minute. Bars indicate SD.

FIG. 4 shows the time course of the increase ratio of ¹³C concentration (Δ¹³C (‰)) in exhaled CO₂ after administration of [1-¹³C]-sodium acetate into the femoral vein. [1-¹³C]-sodium acetate was administered into the femoral vein of nSTZ rats and control rats at 0 minute. Bars indicate SD.

FIG. 5 shows the time course of the increase ratio of ¹³C concentration (Δ¹³C (‰)) in exhaled CO₂ after administration of [1-¹³C]-glutamine into the femoral vein.

[1-¹³C]-glutamine was administered into the femoral vein of nSTZ rats and control rats at 0 minute. Bars indicate SD.

FIG. 6 shows the time course of the increase ratio of ¹³C concentration (Δ¹³C (‰)) in exhaled CO₂ after oral administration of [1,4-¹³C]-succinic acid. [1,4-¹³C]-succinic acid was administered orally to Buscopan-administered rats and Buscopan-non-administered rats at 0 minute.

### BEST MODE FOR CARRYING OUT THE INVENTION

Hereinbelow, the present invention will be described in detail.

The term “gastric emptying function” used herein refers to the function of the stomach to move its contents to the intestinal tract.

The term “small intestinal absorptive capacity” used herein refers to the function of the small intestine to absorb nutrients and the like from its contents.

The term “metabolite” used herein refers to those compounds produced in the process of metabolism of succinic acid or a pharmaceutically acceptable salt thereof which has been administered into a living body.

The expression “labeled with ¹³C” used herein refers to such succinic acid in which the abundance of ¹³C atom has become higher than the natural abundance of ¹³C atom in succinic acid as a result of replacement of at least one carbon atom in succinic acid with ¹³C atom.

The diagnostic agent of the present invention includes succinic acid labeled with ¹³C or a pharmaceutically acceptable salt thereof ¹³C atoms may be present in the major chain or a side chain of succinic acid. Succinic acid labeled with ¹³C is hereinafter referred to as “¹³C-labeled succinic acid”.

The ¹³C-labeled succinic acid used in the present invention may be a commercial product.

Examples of ¹³C-labeled succinic acid include, but are not limited to, [1,4-¹³C] succinic acid, [1-¹³C] succinic acid, [2,3-¹³C] succinic acid, [2-¹³C] succinic acid and [U-¹³C] succinic acid.

In the present invention, a commercial ¹³C-labeled succinic acid may be used without any processing.

The above-described ¹³C-labeled succinic acid may be obtained in the form of salts. Examples of such salts include, but are not limited to, salts formed with alkali metals such as sodium and potassium; salts formed with alkaline earth metals such as calcium; and salts formed with organic amines such as ammonium ethanol amine, triethylamine and dicyclohexylamine may be enumerated.

The diagnostic agent of the present invention may be formulated into a preparation (such as tablets, capsules, powders, granules or liquid preparations) by using the ¹³C-labeled succinic acid or a pharmaceutically acceptable salt alone or in combination with excipients or carriers. As excipients and carriers, those which are conventionally used in the art and pharmaceutically acceptable may be used. Their types and compositions may be varied appropriately. For example, as a liquid carrier, water may be used. As solid carriers, saccharides such as lactose, white sugar and glucose; starches such as potato starch and corn starch; and cellulose derivatives such as crystalline cellulose may be used. Lubricants such as magnesium stearate; binders such as gelatin and hydroxypropyl cellulose; and disintegrants such as carboxymethyl cellulose may also be added. Further, antioxidants, coloring agents, flavoring agents, preservatives and the like may also be added. In liquid preparations, generally, preferable carriers are sterilized water, physiological saline and various buffers. It is also possible to use the diagnostic agent of the present invention as a freeze-dried preparation.

The content of the ¹³C-labeled succinic acid or a pharmaceutically acceptable salt thereof in a preparation is usually 1-100% by weight, preferably 50-100% by weight, though the content varies depending on the type of the preparation. For example, in the case of liquid preparations, the content of the ¹³C-labeled succinic acid or a pharmaceutically acceptable salt thereof in the preparation is preferably 1-100% by weight. In the case of capsules, tablets, granules or powders, the content of the ¹³C-labeled succinic acid or a pharmaceutically acceptable salt thereof in the preparation is usually about 10-100% by weight, preferably 50-100% by weight, and the remaining moiety is a carrier.

The dose of the diagnostic agent of the present invention should be sufficient to confirm increase in ¹³C concentration in the exhaled CO₂ after administration, increase in ¹³C-labeled succinic acid concentration (content) in blood after administration, or metabolites derived from the administered compound. Although the dose varies depending on the age and body weight of the patient and the purpose of the test, the dose per administration for an adult is about 0.1-20 mg/kg body weight (converted to the amount of active ingredient).

Tests using the diagnostic agent of the present invention may be performed as a breath test in which the ¹³C-labeled succinic acid or a pharmaceutically acceptable salt thereof is administered to a subject and then ¹³CO₂ discharged into the breath after the administration is measured. It is also possible to use a plurality of species of the ¹³C-labeled succinic acids or pharmaceutically acceptable salts thereof in combination. In a specific example where a ¹³C-labeled compound is used, ¹³C concentration in the exalted CO₂ after administration is measured, and then diagnosis of gastric emptying function or small intestinal absorptive capacity is performed using data such as the increase ratio of ¹³C concentration (Δ¹³C (‰)) in exhaled CO₂ at a specific time point (e.g., 1 hour, 2 hours, 3 hours, etc.) after the administration, or integration or time course (slope at the rising phase, change in slope, peak time, etc.) of the increase ratio of ¹³C concentration (Δ¹³C (‰)) in exhaled CO₂ by a specific time point after the administration. Alternatively, after administration of the ¹³C-labeled succinic acid or a pharmaceutically acceptable salt thereof to a subject, a test for measuring the ¹³C-labeled succinic acid or metabolites in the blood the may be performed. Further, a plurality of species of the ¹³C-labeled succinic acids or pharmaceutically acceptable salts thereof may be used in combination. Specifically, the concentration (content) of ¹³C-labeled succinic acid or the concentration (content) of a metabolite in the blood after administration is measured. Then, diagnosis of gastric emptying function or small intestinal absorptive capacity is performed from data such as the increase ratio of ¹³C-labeled succinic acid concentration (content) or a metabolite concentration (content) in the blood at a specific time point (e.g., 30 min or 1 hour) after the administration, or integration or time course (slope at the rising phase, change in slope, peak time, etc.) of ¹³C-labeled succinic acid concentration (content) or a metabolite concentration (content) in the blood by a specific time point after the administration.

Measurement of ¹³C-labeled succinic acid concentration (content) or a metabolite concentration (content) in the blood may be performed by methods such as colorimetry, fluorography, a chemical method with ninhydrin or the like, an enzymatic chemical measurement method, a method using a microorganism, chromatography, gas chromatograph-mass spectrometer method (GC-MS), infrared spectrophotometry, mass spectrometry, photoelectric acoustic spectrometry or NMR (nuclear magnetic resonance) method, using total blood, serum or plasma as it is or after operations for separation/purification.

Measurement of ¹³CO₂ in the breath may be performed by methods such as gas chromatograph-mass spectrometer method (GC-MS), infrared spectrophotometry, mass spectrometry, photoelectric acoustic spectrometry or NMR (nuclear magnetic resonance) method.

With the diagnostic agent of the present invention, it is possible to examine whether or not abnormalities are observed in gastric emptying or small intestinal absorption.

Examples of pathologies in which gastric emptying is delayed include, but are not limited to, diabetes, functional dyspepsia and reflux esophagitis. Delay in gastric emptying is also a problem in patients who underwent a gastrointestinal surgery. It has been elucidated that functional dyspepsia is a pathology caused by dysfunction in gastric emptying.

Examples of pathologies in which abnormalities are observed in small intestinal absorptive capacity include, but are not limited to, small intestinal epithelium disorder caused by fasting, drug-induced enteritis caused by administration of drugs such as anticancer agents or non-steroidal anti-inflammatory drugs NSAIDs), short bowel syndrome and irritable bowel syndrome. It is believed that evaluation of small intestinal absorptive capacity is useful in judging anastomotic leak in a patient who underwent a surgery (especially, gastrointestinal surgery) or judging the timing of shifting from parenteral nutrition to enteral or oral feeding.

Detecting abnormalities in gastric emptying and small intestinal absorption by tests using the diagnostic agent of the present invention leads to early diagnosis of the above-listed pathologies.

The site of metabolism of succinic acid is mitochondria, which are present in every cell. Therefore, those tests using the diagnostic agent of the present invention are less affected by metabolism than conventional technologies for evaluating gastric emptying and small intestinal absorption.

Since succinic acid has a small molecular weight, a small dose of the diagnostic agent may be sufficient for diagnosis.

It has been confirmed in a single administration toxicity test on rat that succinic acid does not cause abnormalities in rats at a dose level of 2000 mg/kg (disodium succinate hexahydrate) (Chemical Toxicity Database in The Databases of Chemicals, Global Information Network on Chemicals). Thus, the diagnostic agent of the present invention is highly safe.

Hereinbelow, the present invention will be described in more detail with reference to the following Examples. However, the scope of the present invention is not limited by these Examples. Unless otherwise specified, amino acids used in the following Examples are in the L-configuration.

### EXAMPLES

### Example 1

### ¹³C-labeled Succinic Acid Breath Test/Changes Depending on the Distance from the Pylorus

Overnight-fasted Wistar male rats (8 week-old; Nippon Charles River) were subjected to a laparotomy under anesthesia and treated as follows: (1) ligated at 3 cm from the pylorus (n=1), (2) ligated at 6 cm from the pylorus (n=1), and (3) not ligated (no treatment) (n=2).

[1,4-¹³C]-succinic acid (ICON) was administered into the duodenum upstream of the ligation at 2 mg/ml/kg. The breath after the administration was collected. For rats of (1) and (2) above, the breath was collected at intervals of 2 minutes over 16 minutes to thereby examine the time course of increase ratio of ¹³C concentration (Δ¹³C (‰)) in exhaled CO₂. For rats of (3) above, the breath was collected at intervals of 1 minute over 15 minutes to thereby examine the time course of increase ratio of ¹³C concentration (Δ¹³C (‰)) in exhaled CO₂.

The increase ratio of ¹³C concentration (Δ¹³C (‰)) in exhaled CO₂ was measured as described below. Briefly, the breath was collected at a rate of about 100 to 300 ml/min using a stroke pump (Variable Stroke Pump VS-500; Shibata Kagaku Kogyo), and the CO₂ concentration therein was maintained at about 3%. A Perma Pure Drier (MD-050-12P; Perma Pure INC.) was set between the rat holder and the stroke pump to remove moisture from the breath. When the CO₂ concentration was stabilized, the rat under anesthesia was subjected to a laparotomy. After ligation of the pylorus, [1,4-¹³C]-succinic acid solution was injected into the duodenum with an injection syringe (1 ml) to thereby achieve administration into the duodenum. A breath sample (15 ml) collected with the syringe was sealed in a vacuum vial (10 ml) and subjected to automated analysis by GC-MS (Breath MAT) [FinniganMAT]. Δ¹³C (‰) was calculated from δ¹³C values which are ¹³C values of the breath sample (i.e. differences from the standard substance PDB) using the following formula.

Δ¹³C (‰)=(δ¹³C)t min−(δ¹³ C) 0 min (Formula 1)

### <Breath MAT Measuring Conditions>

Apparatus: Breath MAT plus (Finnigan)
   Carrier gas: He
   Measured ions: m/z=44, 45, 46

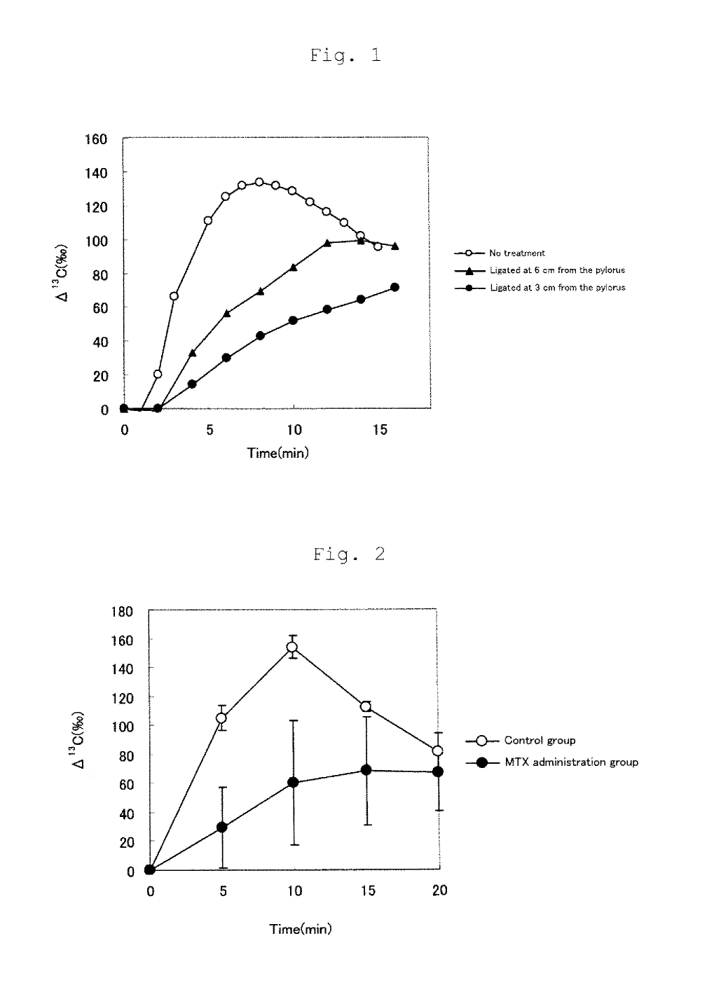

The results are shown in FIG. 1. In (3) where rats were not ligated, Δ¹³C rapidly increased after administration, reached 130‰ at 6 minutes, retained a similar value until 8 minutes, and then decreased to 95‰ at 15 minutes almost linearly.

On the other hand, in (1) and (2) where rats were ligated at 3 cm and 6 cm from the pylorus, respectively, Δ¹³C value continued to increase up to 71‰ for (1) and 96‰ for (2) at 16 minutes. In (1) where the rat was ligated at 3 cm from the pylorus, Δ¹³C at 4 minutes was 14‰ and Δ¹³C at 6 minutes was 30‰. In (2) where the rat was ligated at 6 cm from the pylorus, Δ¹³C at 4 minutes was 33‰ and Δ¹³C at 6 minutes was 57‰, both values being 2 times larger than the corresponding values in (1). Thus, Δ¹³C values at 4 minutes and 6 minutes were reflecting the distance from the pylorus.

Comparison with (3) where rats were not ligated also confirmed that Δ¹³C decreases when the surface area is smaller.

### Example 2

### ¹³C-labeled Succinic Acid Breath Test/Changes Depending on the Presence or Absence of Small Intestinal Disorder

Falling out of small intestinal villi was induced in Wistar male rats (9 week-old; n=4; Nippon Charles River) by repeatedly injecting injectable methotrexate (MTX; Takeda Chemical Industries) dissolved in physiological saline between the back and the skull subcutaneously at 2.5 mg/kg/day (see J. Nutr. Vol. 126, p. 2519 (1996); Scand. J. Gastroenterol. Vol. 39 p. 1015 (2004)). The conditions of the repeated administration were 3 day administration, 2 day interval and 3 day administration (total administration period: 6 days) (MTX administration group; n=4). Non-administration group (n=2) was also tested as control group.

On day 9 after the start of MTX administration, [1,4-¹³C]-succinic acid (ICON) was administered into the duodenum of overnight-fasted rats under anesthesia at 2 mg/ml/kg to perform a breath test.

The increase ratio of ¹³C concentration (Δ¹³C (‰)) in exhaled CO₂ after administration was measured at intervals of 5 minutes to examine the time course for 20 minutes.

The results are shown in FIG. 2.

In the control group (non-administration group), Δ¹³C rapidly increased after administration, reached 154‰ at 10 minutes and then decreased to 81‰ at 20 minutes almost linearly.

On the other hand, in MTX administration group, Δ¹³C gently increased and was 68±37‰ at 15 minutes. Although some dispersion was observed between individuals, the degree of decrease was large compared to the control group. Thus, significant difference was recognized.

### Experimental Example 1

### Preparation of Diabetic Model Rats (nSTZ Group)

Male Sprague-Dawley (SD) rats were purchased from Nippon Charles River. For new born rats, their foster parents were purchased together, These rats were raised at 23±2° C. under 55±10% humidity until the time of use.

Impaired insulin secretion-type diabetes was induced by administering streptozocin (STZ) (Sigma) to neonatal rats (“Cell Engineering” special issue Medical Experiment Manual Series: Diabetes Research Strategy, Susumu Seino & Yoshikazu Oka (Eds.), published by Shujunsha). Briefly, STZ was administered to 2-day postnatal rats subcutaneously at a dose of 90 mg/kg. STZ was dissolved in citrate buffer (pH 4.5) in advance, and administration was completed within 5 minutes after the dissolving. Two days thereafter, a blood sample was collected from the heart and subjected to measurement of casual plasma glucose with Terumo Mediace (blood glucose measuring set). Individuals showing a level of 275 mg/dl or more were selected. In these rats, casual plasma glucose begins to increase 5 weeks after the administration of STZ. Seven weeks after the administration, almost all rats show a high level of casual plasma glucose. However, rise in fasting plasma glucose is slight and they show an almost normal level.

### Experimental Example 2

### ¹³C-labeled Succinic Acid Breath Test/Comparison between Diabetic Model Rats (nSTZ Group) and Control Group

[1,4-¹³C]-succinic acid (ICON) dissolved in physiological saline was administered to non-treated control group (8 week-old; n=4) and nSTZ group (8 week-old; n=4) prepared in Experimental Example 1 at 4 mg/ml/kg from the femoral vein under anesthesia to perform a breath test.

The time course of the increase ratio of ¹³C concentration (Δ¹³C (‰)) in exhaled CO₂ after administration was examined for 20 minutes, and plotted every 5 minutes.

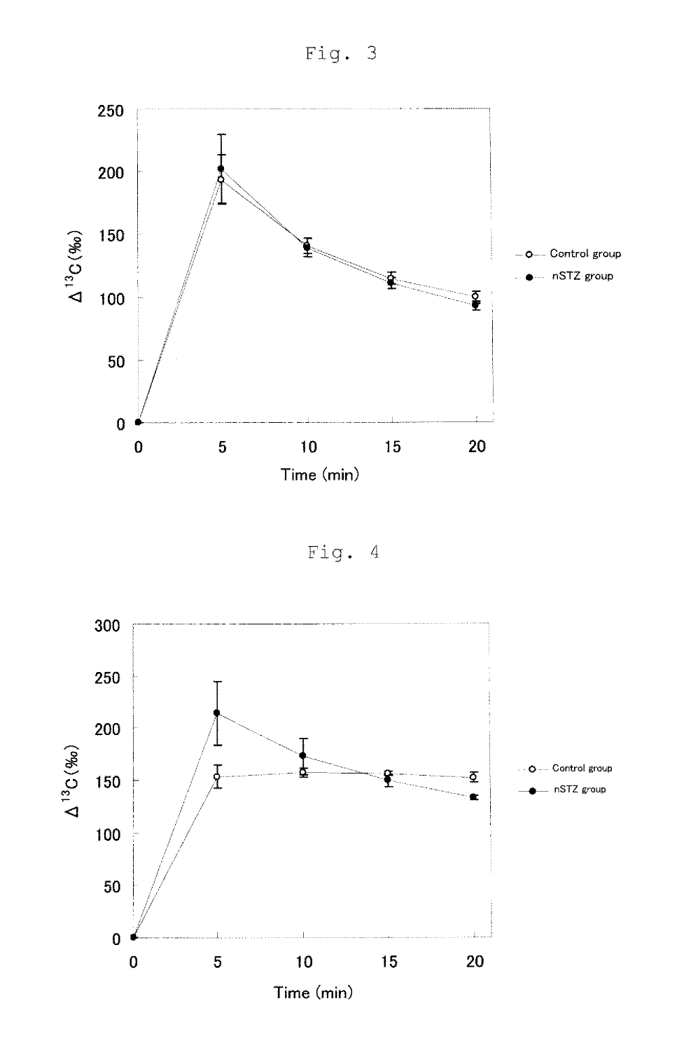

The results are shown in FIG. 3.

In both control group and nSTZ group, Δ¹³C rapidly increased after administration. At 5 minutes, control group reached 193±20‰ and nSTZ group reached 202±28‰. Then, both groups turned to decrease; at 20 minutes, Δ¹³C in control group was 99±4‰ and Δ¹³C in nSTZ group was 93±4‰. Thus, no difference was observed between control group and nSTZ group in the 20 minutes succinic acid breath test.

### Experimental Example 3

### ¹³C-labeled Sodium Acetate Breath Test/Comparison between Diabetic Model Rats (nSTZ Group) and Control Group

[1-¹³C]-sodium acetate (Mass Trace) dissolved in physiological saline was administered to non-treated control group (n=4) and nSTZ group (n=4) prepared in Experimental Example 1 at 10 mg/ml/kg from the femoral vein under anesthesia to perform a breath test.

The time course of the increase ratio of ¹³C concentration (Δ¹³C (‰)) in exhaled CO₂ after administration was examined for 20 minutes, and plotted every 5 minutes.

The results are shown in FIG. 4.

In control group, Δ¹³C reached 154±11‰ at 5 minutes after the administration and retained an almost similar level until 20 minutes (153±5‰). On the other hand, in nSTZ group, Δ¹³C rapidly increased after the administration reached 214±30‰ at 5 minutes, then turned to decrease and was 133±2‰ at 20 minutes. Significant difference (p<0.005) was recognized between control group and nSTZ group in the values at 5 minutes and at 20 minutes.

### Experimental Example 4

### ¹³C-labeled Glutamine Breath Test/Comparison between Diabetic Model Rats (nSTZ Group) and Control Group

[1-¹³C]-glutamine (Mass Trace) dissolved in physiological saline was administered to non-treated control group (8 week-old; n=4) and nSTZ group (8 week-old; n=3) prepared in Experimental Example 1 at 20 mg/ml/kg from the femoral vein under anesthesia to perform a breath test.

The time course of the increase ratio of ¹³C concentration (Δ¹³C (‰)) in exhaled CO₂ after administration was examined for 20 minutes, and plotted every 5 minutes.

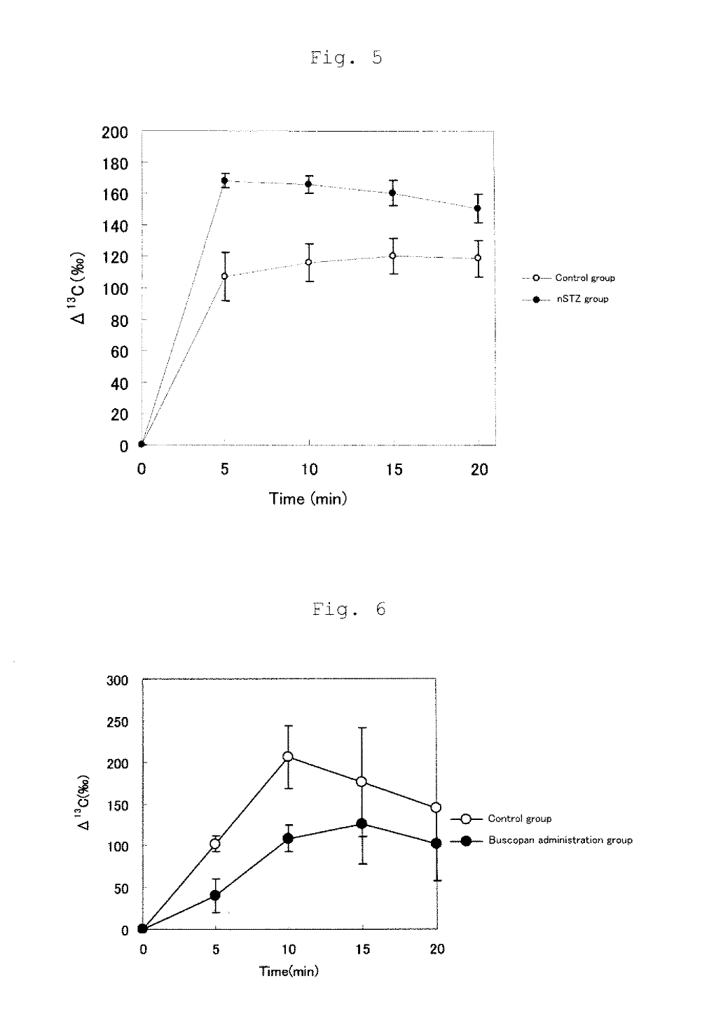

The results are shown in FIG. 5.

In control group, Δ¹³C reached 107±15‰ at 5 minutes after the administration and retained an almost similar level until 20 minutes (119±12‰). On the other hand, in nSTZ group, Δ¹³C reached 168±4‰ at 5 minutes after the administration and retained an almost similar level until 20 minutes (151±9‰). Thus, nSTZ group always showed a higher level than control group in the 20 minutes breath test.

Therefore, it is possible to diagnose small intestinal absorptive capacity by a breath test after the administration of [1,4-¹³C]-succinic acid into the duodenum. It was also found that the breath test using [1,4-¹³C]-succinic acid is not affected by diabetes but breath tests using other carboxylic acid or a salt thereof (sodium acetate, glutamine) are affected by diabetes.

From the results of the above-described Examples and Experimental Examples, the principle of ¹³C-succinic acid breath test is considered as follows. The administered ¹³C-succinic acid is not absorbed in the stomach, but absorbed in the small intestine and metabolized in intracellular mitochondria to produce ¹³CO₂. The ¹³CO₂ after the administration of ¹³C-succinic acid decreases when gastric emptying speed is low or when the small intestinal surface area is small. The ¹³CO₂ is not easily affected by metabolic diseases such as diabetes.

### Example 3

### ¹³C-labeled Succinic Acid Breath Test/Changes Depending on the Presence or Absence of Inhibition of Gastric Motility

¹³C-succinic acid breath test was performed on rats (n=5) who's gastric motility was inhibited by administration of Buscopan (a drug used for inhibiting gastric motility at the time of endoscopy or the like) and non-treated control rats (n=4). As a result, a decrease in ¹³CO₂ in the breath was recognized in Buscopan administration group.
Method: Buscopan (5 mg/ml/kg; Buscopan injection (20 mg/ml; Nippon Boehringer Ingelheim)) diluted 4-fold with physiological saline was administered intraperitoneally into overnight-fasted Wistar rats (7 week-old; male). After leaving them for 15 minutes, ¹³C-succinic acid ([1,4-¹³C]-succinic acid manufactured by CIL) was administered orally (5 mg/5 ml/kg) to start a breath test. Buscopan non-administered group was provided as control group. After the start of the test, breath samples were collected at intervals of 5 minutes for 20 minutes. Then, the time course of the increase ratio of ¹³CO₂ in the breath (Δ¹³C) was examined with Breath MAT (Finnigan) (FIG. 6).
Results: While Δ¹³C in control group (n=4) was 102±10‰ at 5 minutes, 206±37‰ at 10 minutes and 176±65‰ at 15 minutes, Δ¹³C in Buscopan administration group (n=5) was 40±20‰ at 5 minutes, 109±16‰ at 10 minutes, 126±48‰ at 15 minutes and 102±43‰ at 20 minutes. Significant difference (p<0.05) was recognized in the values at 5 minutes and 10 minutes. Although no significant difference was recognized in the values at 15 minutes and 20 minutes, Buscopan administration group showed a tendency of decrease.

Since Δ¹³C decreased as a result of administration of Buscopan which causes inhibition of gastric motility, we can say that it is possible to detect delayed gastric emptying by ¹³C-succinic acid breath test.

### Formulation Example 1

### Tablet

Powders of [1,4-¹³C]-succinic acid (100 mg), lactose (79 mg), corn starch (20 mg) and magnesium stearate (1 mg) are mixed sufficiently and shaped into tablets, each weighing 200 mg. If necessary, these tablets may be coated with sugar or a film.

All publications, patents and patent applications cited herein are incorporated herein by reference in their entirety.

### INDUSTRIAL APPLICABILITY

According to the present invention, diagnosis of gastric emptying function and/or small intestinal absorptive capacity has become possible which imposes less burden to subjects and which is capable of obtaining objective test results in a short period of time. The diagnostic agent of the present invention is cheap, and since it has no adverse effect, it can be used safely.

1. A diagnostic agent for evaluating gastric emptying function and/or small
intestinal absorptive capacity, comprising succinic acid labeled with a stable isotope ¹³C or a pharmaceutically acceptable salt thereof.

2. The diagnostic agent according to claim 1, wherein the succinic acid is selected from the group consisting of [1,4-¹³C] succinic acid, [1-¹³C] succinic acid, [2,3-¹³C] succinic acid, [2-¹³C] succinic acid and [U-¹³C] succinic acid.

3. The diagnostic agent according to claim 1 or 2, which is a preparation for oral administration.

4. The diagnostic agent according to claim 1, which is for use in a breath test where ¹³CO₂ discharged into the breath after administration is measured.

5. The diagnostic agent according to claim 1, which detects diseases associated with abnormalities in gastric emptying function and/or small intestinal absorptive capacity.

6. The diagnostic agent according to claim 5, which detects functional dyspepsia.

7. The diagnostic agent according to claim 5, which detects small intestinal
epithelium disorder caused by fasting, drug-induced enteritis, short bowel syndrome or irritable bowel syndrome.

8. The diagnostic agent according to claim 1, which evaluates the gastric emptying function of a patient who underwent a gastrointestinal surgery, a patient with diabetes, a patient with functional dyspepsia or a patient with reflux esophagitis.

9. The diagnostic agent according to claim 1, which is for use in judging anastomotic leak in a patient who underwent a gastrointestinal surgery.

10. The diagnostic agent according to claim 1, which is for use in judging the appropriateness of shifting from parenteral nutrition to enteral nutrition or oral feeding.

11. Use of succinic acid labeled with a stable isotope ¹³C or a pharmaceutically acceptable salt thereof for manufacturing a diagnostic agent for evaluating gastric emptying function and/or small intestinal absorptive capacity.

12. A method of evaluating gastric emptying function and/or small intestinal absorptive capacity, comprising administering to a subject succinic acid labeled with a stable isotope ¹³C or a pharmaceutically acceptable salt thereof.

13. The diagnostic agent according to claim 2, which is for use in a breath test where ¹³CO₂ discharged into the breath after administration is measured.

14. The diagnostic agent according to claim 2, which detects diseases associated with abnormalities in gastric emptying function and/or small intestinal absorptive capacity.

15. The diagnostic agent according to claim 2, which evaluates the gastric emptying function of a patient who underwent a gastrointestinal surgery, a patient with diabetes, a patient with functional dyspepsia or a patient with reflux esophagitis.

16. The diagnostic agent according to claim 2, which is for use in judging anastomotic leak in a patient who underwent a gastrointestinal surgery.

17. The diagnostic agent according to claim 2, which is for use in judging the appropriateness of shifting from parenteral nutrition to enteral nutrition or oral feeding.
